(19) United States
(12) Patent Application Publication
(10) Pub. No.: US 2025/0186725 A1
(43) Pub. Date: Jun. 12, 2025

(54) CRICOTHYROTOMY DEVICE AND USES THEREOF

(71) Applicant: Edward Via College of Osteopathic Medicine, Blacksburg, VA (US)

(72) Inventors: David Kashmer, Blacksburg, VA (US); Fred Rawlins, Blacksburg, VA (US); Kaitlin Dinsmoore, Blacksburg, VA (US); Angela Burns, Blacksburg, VA (US); Jacob Aron, Blacksburg, VA (US); Abby Tissier, Blacksburg, VA (US); Karen Andrawes, Blacksburg, VA (US); Matthew Boss, Blacksburg, VA (US); Christopher Johnson, Blacksburg, VA (US)

(21) Appl. No.: 18/978,427

(22) Filed: Dec. 12, 2024

Related U.S. Application Data
(60) Provisional application No. 63/609,020, filed on Dec. 12, 2023.

(51) Int. Cl. A61M 16/04

(52) U.S. Cl. CPC A61M 16/0402; A61M 2205/0216; A61M 2205/0233; A61M 2205/32; A61M 2210/1032

(57) ABSTRACT

Described in several embodiments herein are cricothyrotomy devices and uses thereof.

### CROSS-REFERENCE TO RELATED APPLICATIONS

This application claims the benefit of and priority to U.S. Provisional Patent Application No. 63/609,020, filed on Dec. 12, 2023, the contents of which is incorporated by reference herein in its entirety.

### TECHNICAL FIELD

The subject matter disclosed herein is generally directed to cricothyrotomy devices and uses thereof.

### BACKGROUND

Cricothyrotomy (also referred to as cricothyroidotomy) is a procedure that involves placement of a tube through an incision in the cricothyroid membrane to establish an airway in a subject. Typically, cricothyrotomy is performed under emergent circumstances. Thus, devices that increase speed and confidence of procedure performance during high stress conditions are warranted.

Citation or identification of any document in this application is not an admission that such a document is available as prior art to the present invention.

### SUMMARY

In some aspects, the devices described herein relate to a cricothyrotomy device including a port including: an elongated cannula portion including a tapered end; a base cannula portion coupled to or integrated with the elongated cannula portion, wherein the base cannula portion is coupled to or integrated with the elongated cannula portion at the end of the elongated cannula portion opposite the tapered end; a port opening in the elongated cannula portion, tapered end, or extending into both, wherein the elongated cannula portion and the base cannula portion are coupled or integrated such that an opening extends from the port opening through the elongated cannula portion and base cannula portion; a port screw thread on the outer surface of the elongated cannula portion at the end of the elongated cannula portion opposite the tapered end and adjacent to the base cannula portion; one or more lock tabs, wherein the one or more lock tabs are positioned on an outer surface of the elongated cannula portion at the end of the port screw thread adjacent to the base cannula portion; and a ring lock configured to couple with the port, the ring lock including: a ring body having a central opening; a ring lock screw thread on the surface of a wall of the ring body facing the central opening, wherein the ring lock screw thread is configured to couple with the port screw thread during use; and one or more notches configured to couple with the one or more lock tabs of the port during use.

In some aspects, the devices described herein relate to a cricothyrotomy device, wherein the port has a length (lₚ) and wherein the length of the port ranges from about 1 cm to about 6 cm.

In some aspects, the devices described herein relate to a cricothyrotomy device, wherein the elongated cannula portion has a length (l_{ec}), wherein the length of the elongated cannula portion (l_{ec}) extends from a tip of the tapered end of the elongated cannula to the base cannula portion and wherein the length of the elongated cannula portion (l_{ec}) ranges from about 0.5 cm to about 5.5 cm (l_{ec}).

In some aspects, the devices described herein relate to a cricothyrotomy device, wherein the opening has a diameter of about 0.1 cm to about 1.5 cm.

In some aspects, the devices described herein relate to a cricothyrotomy device, wherein the port opening has a diameter of about 0.1 cm to about 1.5 cm.

In some aspects, the devices described herein relate to a cricothyrotomy device, wherein a thickness of a wall of the elongated cannula ranges from about 0.1 cm to about 0.5 cm.

In some aspects, the devices described herein relate to a cricothyrotomy device, wherein a length of the tapered end (lₜₑ) ranges from 0.1 cm to about 0.5 cm.

In some aspects, the devices described herein relate to a cricothyrotomy device, wherein the port screw thread has a length (lₚₛₜ) ranging from about 0.5 cm to about 3 cm.

In some aspects, the devices described herein relate to a cricothyrotomy device, wherein an outer diameter of the elongated cannula ranges from about 0.3 cm to about 2.5 cm.

In some aspects, the devices described herein relate to a cricothyrotomy device, wherein the base cannula portion has a width (w_{bc}) ranging from about 0.25 cm to about 2 cm.

In some aspects, the devices described herein relate to a cricothyrotomy device, wherein the base cannula portion has a length (l_{bc}) ranging from about 0.5 cm to about 3 cm.

In some aspects, the devices described herein relate to a cricothyrotomy device, wherein a wall of the base cannula portion has a thickness ranging from about 0.3 cm to about 2.25 cm.

In some aspects, the devices described herein relate to a cricothyrotomy device, wherein an outer diameter of the base cannula portion ranges from about 0.6 cm to about 6 cm.

In some aspects, the devices described herein relate to a cricothyrotomy device, wherein an outer diameter of the ring lock (d_{odr1}) ranges from about 0.6 cm to about 6 cm.

In some aspects, the devices described herein relate to a cricothyrotomy device, wherein a diameter of the central opening (d_{co}) of the ring lock ranges from about 0.3 cm to about 2.5 cm.

In some aspects, the devices described herein relate to a cricothyrotomy device, wherein a width of the ring body ranges (w_{rb}) from about 0.1 cm to about 1.25 cm.

In some aspects, the devices described herein relate to a cricothyrotomy device, wherein the ring body includes an inner wall and an outer wall, wherein a thickness of the inner wall and the outer wall each independently ranges from about 0.1 cm to about 0.25 cm.

In some aspects, the devices described herein relate to a cricothyrotomy device, wherein the ring body includes notch walls, wherein a thickness of the notch walls ranges from about 0.1 cm to about 0.25 cm.

In some aspects, the devices described herein relate to a cricothyrotomy device, wherein the port and the ring lock each independently include or consist of a metal, an alloy, a polymer, silicone, or any combination thereof.

In some aspects, the devices described herein relate to a cricothyrotomy device, wherein the port and the ring lock each independently include or consist of a radio translucent material or a radio opaque material.

Described in several aspects herein are methods of performing a cricothyrotomy comprising coupling the port of the cricothyrotomy device of the present disclosure to the ring lock of the cricothyrotomy device by inserting the tapered end of the port into the central opening of the ring lock and moving the ring lock to the base cannula portion of the port; and inserting the tapered end of the elongated cannula into a neck of the subject such that an airway is established. In some aspects, coupling the port to the ring lock comprises screwing the ring lock screw thread onto the port screw thread.

In some aspects, the insertion of the tapered end is through an incision in a cricothyroid membrane of the subject or wherein insertion of the tapered end forms an incision in the cricothyroid membrane of the subject.

In some aspects, the method further includes inserting one or more guide wires through the opening of the cricothyrotomy device and into a trachea of the subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows one view of one embodiment of a cricothyrotomy device of the present disclosure.

FIG. 2 shows a second view of the embodiment of a cricothyrotomy device of the present disclosure.

FIG. 3 shows a view of one embodiment of a port of the cricothyrotomy device illustrating several exemplary dimensions of the port.

FIG. 4 shows a view of one embodiment of a ring lock of the cricothyrotomy device illustrating several exemplary dimensions of the port.

FIG. 5 shows an image of a prototype of a cricothyrotomy device according to an embodiment of the present disclosure with the collar screwed on the port as would be during use.

FIG. 6 shows an image of a prototype of a device according to an embodiment of the present disclosure with the collar separate from the port as would be before or after use.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Cricothyrotomy (also referred to as cricothyroidotomy) is a procedure that involves placement of a tube through an incision in the cricothyroid membrane to establish an airway in a subject. Typically, cricothyrotomy is performed under emergent circumstances. Thus, devices that increase speed and confidence of procedure performance during high stress conditions are warranted. Although other cricothyrotomy devices are available, they are plagued by slipping out of place during use or are complex making them less than ideal for use in emergent or training situations.

With that said, embodiments disclosed herein can provide cricothyrotomy devices that include a port composed of an elongated cannula that is tapered to a point at one and having a base cannula portion at the end of the elongated cannula opposite the tapered end. The base cannula portion is wider than the elongated cannula portion. There is a port opening at the end of the cannula portion near or extending into the tapered end. The elongated cannula portion and the base cannula portion are configured such that an inner hole extends through elongated cannula portion and base cannula portion such that an opening extends through the port from the base cannula portion to the port opening. The port is configured to receive a ring lock. In operation the ring lock can couple with the port and ensure that the device is secured in place during a cricothyrotomy. The ring lock can also help to ensure proper positioning of the port. The ring lock is composed of a ring having a central opening that is lined with a screw thread. The ring lock can also contain one or more notches along the outer circumference of the ring. The elongated cannula portion can contain a screw thread along the end adjacent to the base cannula portion that is configured to receive a screw thread on the ring lock. During use the elongated cannula portion can be placed through the central opening on the ring lock via inserting the tapered end first into the central opening on the ring lock. The port and the ring lock can be coupled together by screwing the ring lock onto the screw thread of the port. The one or more notches on the ring lock can engage one or more lock tabs at the on the elongated cannula portion at or near end of the screw thread closest to the base cannula portion.

Other devices, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

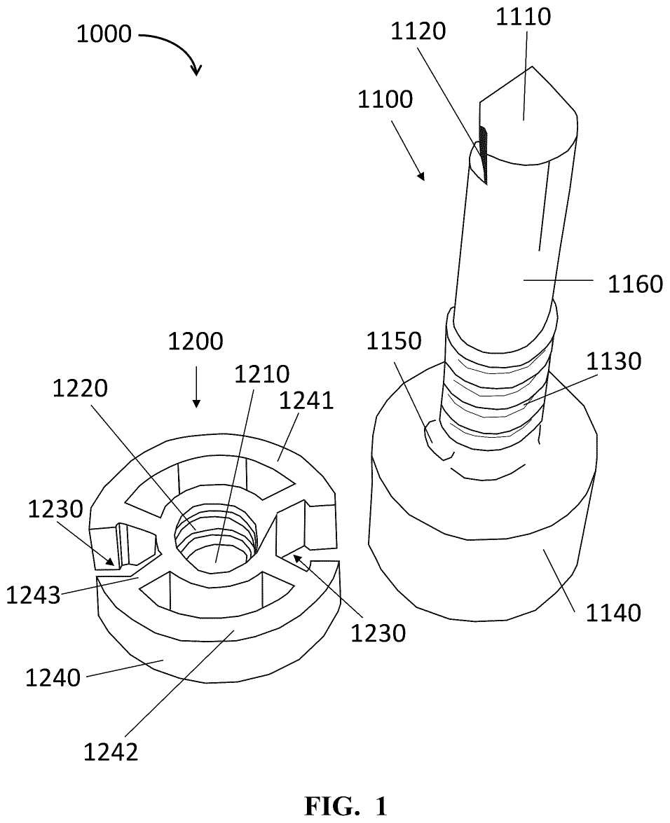

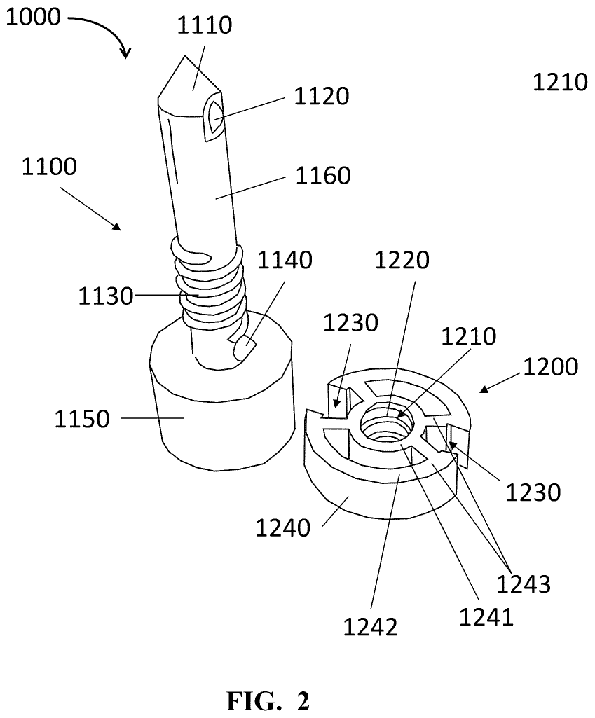

As generally shown in FIGS. 1-2, described herein are embodiments of a cricothyrotomy device 1000 including a port 1100 and a ring lock 1200. In some embodiments, the port 1100 comprises an elongated cannula portion 1160 including a tapered end 1110. In some embodiments, the tapered end is such that the tapered end has a tip that is sharp enough to be inserted through an incision in a subject. In some embodiments, the tip of the tapered end 1110 is sharp enough such that it is capable of making an incision or pierce the skin, muscle, and/or other soft tissues of the subject, such as the cricothyrotomy membrane of the subject In some embodiments, the port 1100 comprises a base cannula portion 1140 coupled to or integrated with the elongated cannula portion 1160, wherein the base cannula portion 1140 is coupled to or integrated with the elongated cannula 1160 portion at the end of the elongated cannula portion 1160 opposite the tapered end 1110. The port 1100 can have a port opening 1120. The port opening 1120 can be any suitable size or shape. In some embodiments, the port opening 1120 is a circle, an oval, a slit, or a square. In some embodiments port opening 1120 can be in the elongated cannula portion 1160. The port opening 1120 can be in the tapered end 1110. In some embodiments, the port opening can extend into both in the elongated cannula portion 1160 and the tapered end 1110. In some embodiments, the elongated cannula portion 1160 and the base cannula portion 1140 are coupled or integrated such that an opening extends from the port opening 1120 through the middle elongated cannula portion 1160 such that the elongated cannula portion 1160 has walls having a thickness. The base cannula portion 1140 can have an opening in substantially the middle of the base cannula 1140. The base cannula portion 1140 and the elongated cannula portion 1160 are coupled or otherwise integrated such that the opening of the base portion continues through the middle of the base cannula portion 1140 such that there is a continuous opening through the port from an end of the base cannula portion 1140 to the port opening 1120. In some embodiments, the port 1100 is a single continuous piece. In some embodiments, the base cannula portion 1140 is a separate component from the elongated cannula portion 1160 and both are configured to couple to each other prior to use to form the port 1100.

In some embodiments, the port 1100 has a port screw thread 1130 on the outer surface of the elongated cannula portion 1130. In some embodiments, the port screw thread 1130 is positioned substantially at or near the end of the elongated cannula portion opposite the tapered end 1120 and near or adjacent to the base cannula portion 1140. In some embodiments, the screw thread extends about 10% to about 50% of the length of the elongated cannula portion 1160. In some embodiments, the screw thread extends about 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.1%, 30.2%, 30.3%, 30.4%, 30.5%, 30.6%, 30.7%, 30.8%, 30.9%, 31%, 31.1%, 31.2%, 31.3%, 31.4%, 31.5%, 31.6%, 31.7%, 31.8%, 31.9%, 32%, 32.1%, 32.2%, 32.3%, 32.4%, 32.5%, 32.6%, 32.7%, 32.8%, 32.9%, 33%, 33.1%, 33.2%, 33.3%, 33.4%, 33.5%, 33.6%, 33.7%, 33.8%, 33.9%, 34%, 34.1%, 34.2%, 34.3%, 34.4%, 34.5%, 34.6%, 34.7%, 34.8%, 34.9%, 35%, 35.1%, 35.2%, 35.3%, 35.4%, 35.5%, 35.6%, 35.7%, 35.8%, 35.9%, 36%, 36.1%, 36.2%, 36.3%, 36.4%, 36.5%, 36.6%, 36.7%, 36.8%, 36.9%, 37%, 37.1%, 37.2%, 37.3%, 37.4%, 37.5%, 37.6%, 37.7%, 37.8%, 37.9%, 38%, 38.1%, 38.2%, 38.3%, 38.4%, 38.5%, 38.6%, 38.7%, 38.8%, 38.9%, 39%, 39.1%, 39.2%, 39.3%, 39.4%, 39.5%, 39.6%, 39.7%, 39.8%, 39.9%, 40%, 40.1%, 40.2%, 40.3%, 40.4%, 40.5%, 40.6%, 40.7%, 40.8%, 40.9%, 41%, 41.1%, 41.2%, 41.3%, 41.4%, 41.5%, 41.6%, 41.7%, 41.8%, 41.9%, 42%, 42.1%, 42.2%, 42.3%, 42.4%, 42.5%, 42.6%, 42.7%, 42.8%, 42.9%, 43%, 43.1%, 43.2%, 43.3%, 43.4%, 43.5%, 43.6%, 43.7%, 43.8%, 43.9%, 44%, 44.1%, 44.2%, 44.3%, 44.4%, 44.5%, 44.6%, 44.7%, 44.8%, 44.9%, 45%, 45.1%, 45.2%, 45.3%, 45.4%, 45.5%, 45.6%, 45.7%, 45.8%, 45.9%, 46%, 46.1%, 46.2%, 46.3%, 46.4%, 46.5%, 46.6%, 46.7%, 46.8%, 46.9%, 47%, 47.1%, 47.2%, 47.3%, 47.4%, 47.5%, 47.6%, 47.7%, 47.8%, 47.9%, 48%, 48.1%, 48.2%, 48.3%, 48.4%, 48.5%, 48.6%, 48.7%, 48.8%, 48.9%, 49%, 49.1%, 49.2%, 49.3%, 49.4%, 49.5%, 49.6%, 49.7%, 49.8%, 49.9%, to/or 50% of the length of the elongated cannula portion 1160.

In some embodiments, the elongated cannula portion 1160 comprises one or more lock tabs 1150. The one or more lock tabs can be positioned on the outer surface of the elongated cannula portion 1160 at the end of the port screw thread 1130 near or adjacent to the base cannula portion 1140.

The ring lock 1200 can be configured to couple with the port 1100. In some embodiments, the ring lock 1200 includes a ring body 1240 having a central opening 1210. In some embodiments, the ring body 1240 has a ring lock screw thread 1220 on the surface of a wall of the ring body 1240 facing the central opening 1210. The ring lock screw thread 1220 can be configured to couple with the port screw thread 1130 during use. The one or more notches 1230 can be configured to couple with the one or more lock tabs 1150 of the port 1100 during use. During use, as the ring lock 1200 is screwed onto the port 1100 the one or more lock tabs 1150 can fit into the one or more notches 1230 thus coupling the one or more notches 1230 with the one or more 1150. This can also lock the ring lock 1200 into place on the port 1100.

In some embodiments, the cricothyrotomy device 1000 is configured for single use. In some embodiments, the cricothyrotomy device 1000 is configured for multiple uses. In some embodiments, the cricothyrotomy device 1000 can be sterilized.

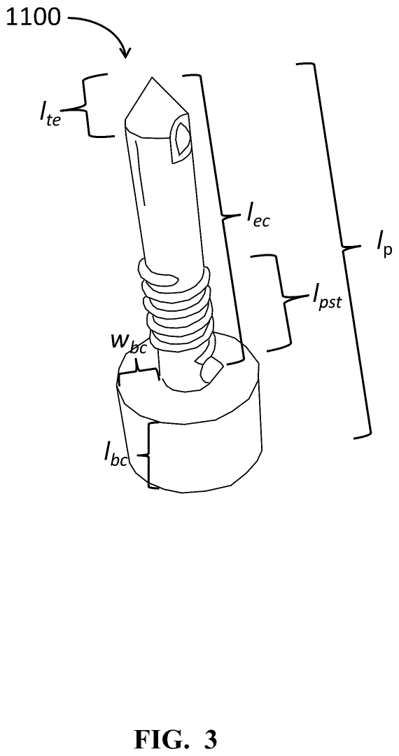

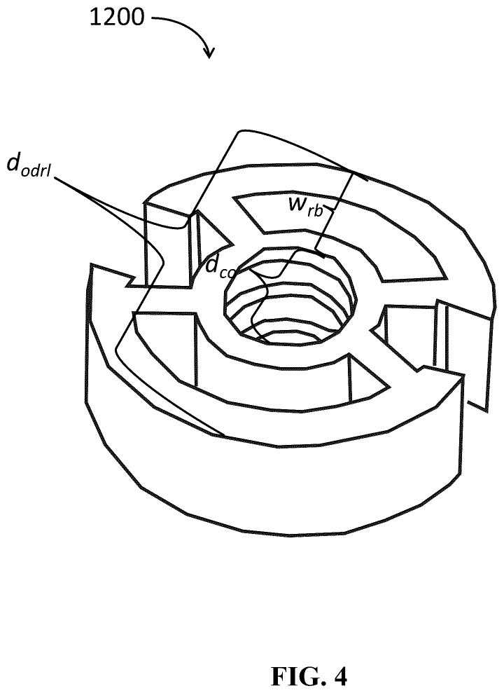

FIGS. 3-4 illustrate several dimensions of the port 1100 and ring lock 1200, respectively. In some embodiments, the port 1100 has a length (lₚ) and wherein the length of the port (lₚ) is or ranges from about 1 cm to about 6 cm. In some embodiments, lₚ is about 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, 5.5 cm, 5.6 cm, 5.7 cm, 5.8 cm, 5.9 cm, or 6 cm. In some embodiments, lₚ ranges from about 1 cm, to about 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, 5.5 cm, 5.6 cm, 5.7 cm, 5.8 cm, 5.9 cm, or 6 cm or ranges between any other range of values therein. In some embodiments, the elongated cannula portion 1160 has a length (l_{ec}), wherein the length of the elongated cannula portion (l_{ec}) extends from a tip of the tapered end 1110 of the elongated cannula portion 1160 to the base cannula portion 1140 and wherein the length of the elongated cannula portion (l_{ec}) ranges from about 0.5 cm to about 5.5 cm. In some embodiments, l_{ec} is about 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, or 5.5 cm. In some embodiments, l_{ec} ranges from about 0.5 cm, to 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, or 5.5 cm In some embodiments, the opening of the base cannula portion 1140 has a diameter of about 0.1 cm to about 1.5 cm. In some embodiments, the opening of the base cannula portion 1140 has a diameter of about 0.1 cm, 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, or 1.5 cm. In some embodiments, the opening of the base cannula portion ranges from about 0.1 cm to 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, or 1.5 cm. In some embodiments, the port opening 1120 has a diameter or longest dimension of about 0.1 cm to about 1.5 cm. In some embodiments, the port opening is about 0.1 cm, 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, or 1.5 cm. In some embodiments, the port opening 1120 has a diameter or longest dimension ranging from about 0.1 cm to 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, or 1.5 cm.

In some embodiments, a thickness of a wall of the elongated cannula 1160 ranges from about 0.1 cm to about 0.5 cm. In some embodiments, the thickness of a wall of the elongated cannula 1160 is about 0.1 cm, 0.2 cm, 0.3 cm, 0.4 cm, or 0.5 cm. In some embodiments, the thickness of a wall of the elongated cannula 1160 ranges from about 0.1 cm to 0.2 cm, 0.3 cm, 0.4 cm, or 0.5 cm. It will be appreciated that the elongated cannula portion 1160 inherently has a wall surrounding the opening extending through the elongated cannula portion 1160.

In some embodiments, the length of the tapered end (lₜₑ) ranges from 0.1 cm to about 0.5 cm. The taper of the tapered end can have any suitable angle. In some embodiments, the angle of the taper is about 10 degrees to about 45 degrees. In some embodiments, the angle of the taper is about 10 degrees, to 10.5 degrees, 11 degrees, 11.5 degrees, 12 degrees, 12.5 degrees, 13 degrees, 13.5 degrees, 14 degrees, 14.5 degrees, 15 degrees, 15.5 degrees, 16 degrees, 16.5 degrees, 17 degrees, 17.5 degrees, 18 degrees, 18.5 degrees, 19 degrees, 19.5 degrees, 20 degrees, 20.5 degrees, 21 degrees, 21.5 degrees, 22 degrees, 22.5 degrees, 23 degrees, 23.5 degrees, 24 degrees, 24.5 degrees, 25 degrees, 25.5 degrees, 26 degrees, 26.5 degrees, 27 degrees, 27.5 degrees, 28 degrees, 28.5 degrees, 29 degrees, 29.5 degrees, 30 degrees, 30.5 degrees, 31 degrees, 31.5 degrees, 32 degrees, 32.5 degrees, 33 degrees, 33.5 degrees, 34 degrees, 34.5 degrees, 35 degrees, 35.5 degrees, 36 degrees, 36.5 degrees, 37 degrees, 37.5 degrees, 38 degrees, 38.5 degrees, 39 degrees, 39.5 degrees, 40 degrees, 40.5 degrees, 41 degrees, 41.5 degrees, 42 degrees, 42.5 degrees, 43 degrees, 43.5 degrees, 44 degrees, 44.5 degrees, or 45 degrees. In some embodiments, the angle of the taper ranges from about 10 degrees, 10.5 degrees, 11 degrees, 11.5 degrees, 12 degrees, 12.5 degrees, 13 degrees, 13.5 degrees, 14 degrees, 14.5 degrees, 15 degrees, 15.5 degrees, 16 degrees, 16.5 degrees, 17 degrees, 17.5 degrees, 18 degrees, 18.5 degrees, 19 degrees, 19.5 degrees, 20 degrees, 20.5 degrees, 21 degrees, 21.5 degrees, 22 degrees, 22.5 degrees, 23 degrees, 23.5 degrees, 24 degrees, 24.5 degrees, 25 degrees, 25.5 degrees, 26 degrees, 26.5 degrees, 27 degrees, 27.5 degrees, 28 degrees, 28.5 degrees, 29 degrees, 29.5 degrees, 30 degrees, 30.5 degrees, 31 degrees, 31.5 degrees, 32 degrees, 32.5 degrees, 33 degrees, 33.5 degrees, 34 degrees, 34.5 degrees, 35 degrees, 35.5 degrees, 36 degrees, 36.5 degrees, 37 degrees, 37.5 degrees, 38 degrees, 38.5 degrees, 39 degrees, 39.5 degrees, 40 degrees, 40.5 degrees, 41 degrees, 41.5 degrees, 42 degrees, 42.5 degrees, 43 degrees, 43.5 degrees, 44 degrees, 44.5 degrees, or 45 degrees.

In some embodiments, the port screw thread has a length (lₚₛₜ) ranging from about 0.5 cm to about 3 cm. In some embodiments, lₚₛₜ is 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, or 3.0 cm. In some embodiments, lₚₛₜ ranges from about 0.5 cm to 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, or 3.0 cm.

In some embodiments, an outer diameter of the elongated cannula portion 1160 ranges from about 0.3 cm to about 2.5 cm. In some embodiments, the outer diameter of the elongated cannula portion 1160 is about 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, or 2.5 cm. In some embodiments, the outer diameter of the elongated cannula portion 1160 ranges from about 0.3 cm to 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, or 2.5 cm.

In some embodiments, the base cannula portion 1140 has a width (w_{bc}) ranging from about 0.25 cm to about 2 cm. In some embodiments, w_{bc} is about 0.25 cm, 0.3 cm, 0.35 cm, 0.4 cm, 0.45 cm, 0.5 cm, 0.55 cm, 0.6 cm, 0.65 cm, 0.7 cm, 0.75 cm, 0.8 cm, 0.85 cm, 0.9 cm, 0.95 cm, 1 cm, 1.05 cm, 1.1 cm, 1.15 cm, 1.2 cm, 1.25 cm, 1.3 cm, 1.35 cm, 1.4 cm, 1.45 cm, 1.5 cm, 1.55 cm, 1.6 cm, 1.65 cm, 1.7 cm, 1.75 cm, 1.8 cm, 1.85 cm, 1.9 cm, 1.95 cm, or 2.0 cm. In some embodiments, w_{bc} ranges from about 0.25 cm to 0.3 cm, 0.35 cm, 0.4 cm, 0.45 cm, 0.5 cm, 0.55 cm, 0.6 cm, 0.65 cm, 0.7 cm, 0.75 cm, 0.8 cm, 0.85 cm, 0.9 cm, 0.95 cm, 1 cm, 1.05 cm, 1.1 cm, 1.15 cm, 1.2 cm, 1.25 cm, 1.3 cm, 1.35 cm, 1.4 cm, 1.45 cm, 1.5 cm, 1.55 cm, 1.6 cm, 1.65 cm, 1.7 cm, 1.75 cm, 1.8 cm, 1.85 cm, 1.9 cm, 1.95 cm, or 2.0 cm.

In some embodiments, the base cannula portion has a length (l_{bc}) ranging from about 0.5 cm to about 3 cm. In some embodiments, l_{bc} is about 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, or 3.0 cm. In some embodiments, l_{bc} ranges from about 0.5 cm to 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, or 3.0 cm.

In some embodiments, a wall of the base cannula portion 1140 has a thickness ranging from about 0.3 cm to about 2.25 cm. In some embodiments, the wall of the base cannula portion 1140 has a thickness of about 0.3 cm, 0.35 cm, 0.4 cm, 0.45 cm, 0.5 cm, 0.55 cm, 0.6 cm, 0.65 cm, 0.7 cm, 0.75 cm, 0.8 cm, 0.85 cm, 0.9 cm, 0.95 cm, 1 cm, 1.05 cm, 1.1 cm, 1.15 cm, 1.2 cm, 1.25 cm, 1.3 cm, 1.35 cm, 1.4 cm, 1.45 cm, 1.5 cm, 1.55 cm, 1.6 cm, 1.65 cm, 1.7 cm, 1.75 cm, 1.8 cm, 1.85 cm, 1.9 cm, 1.95 cm, 2 cm, 2.05 cm, 2.1 cm, 2.15 cm, 2.2 cm, 2.25 cm, or 2.25 cm. In some embodiments, the wall of the base cannula portion 1140 has a thickness ranging from about 0.3 cm, 0.35 cm, 0.4 cm, 0.45 cm, 0.5 cm, 0.55 cm, 0.6 cm, 0.65 cm, 0.7 cm, 0.75 cm, 0.8 cm, 0.85 cm, 0.9 cm, 0.95 cm, 1 cm, 1.05 cm, 1.1 cm, 1.15 cm, 1.2 cm, 1.25 cm, 1.3 cm, 1.35 cm, 1.4 cm, 1.45 cm, 1.5 cm, 1.55 cm, 1.6 cm, 1.65 cm, 1.7 cm, 1.75 cm, 1.8 cm, 1.85 cm, 1.9 cm, 1.95 cm, 2 cm, 2.05 cm, 2.1 cm, 2.15 cm, 2.2 cm, 2.25 cm, or 2.25 cm.

In some embodiments, an outer diameter of the base cannula portion 1140 ranges from about 0.6 cm to about 6 cm. In some embodiments, an outer diameter of the base cannula portion 1140 is about 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, 5.5 cm, 5.6 cm, 5.7 cm, 5.8 cm, 5.9 cm, or 6 cm. In some embodiments, an outer diameter of the base cannula portion 1140 ranges from about 0.6 cm to 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, 5.5 cm, 5.6 cm, 5.7 cm, 5.8 cm, 5.9 cm, or 6 cm.

In some embodiments, an outer diameter of the ring lock (d_{odr1}) ranges from about 0.6 cm to about 6 cm. In some embodiments, d_{odr1} is about 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, 5.5 cm, 5.6 cm, 5.7 cm, 5.8 cm, 5.9 cm, or 6 cm. In some embodiments, d_{odr1} ranges from about 0.6 cm to 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, 5 cm, 5.1 cm, 5.2 cm, 5.3 cm, 5.4 cm, 5.5 cm, 5.6 cm, 5.7 cm, 5.8 cm, 5.9 cm, or 6 cm.

In some embodiments, a diameter of the central opening (d_{co}) of the ring lock ranges from about 0.3 cm to about 2.5 cm. In some embodiments, d_{co} is about 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, or 2.5 cm. In some embodiments, d_{co} ranges from about 0.3 cm to 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, or 2.5 cm.

In some embodiments, a width of the ring body (w_{rb}) ranges from about 0.1 cm to about 1.25 cm. In some embodiments, a width of the ring body (w_{rb}) is about 1 cm, 1.01 cm, 1.02 cm, 1.03 cm, 1.04 cm, 1.05 cm, 1.06 cm, 1.07 cm, 1.08 cm, 1.09 cm, 1.1 cm, 1.11 cm, 1.12 cm, 1.13 cm, 1.14 cm, 1.15 cm, 1.16 cm, 1.17 cm, 1.18 cm, 1.19 cm, 1.2 cm, 1.21 cm, 1.22 cm, 1.23 cm, 1.24 cm, or 1.25 cm. In some embodiments, a width of the ring body (w_{rb}) ranges from about 1 cm, 1.01 cm, 1.02 cm, 1.03 cm, 1.04 cm, 1.05 cm, 1.06 cm, 1.07 cm, 1.08 cm, 1.09 cm, 1.1 cm, 1.11 cm, 1.12 cm, 1.13 cm, 1.14 cm, 1.15 cm, 1.16 cm, 1.17 cm, 1.18 cm, 1.19 cm, 1.2 cm, 1.21 cm, 1.22 cm, 1.23 cm, 1.24 cm, or 1.25 cm.

In some embodiments, the ring body includes an inner wall 1241 and an outer wall 1242, wherein a thickness of the inner wall 1241 and the outer wall 1242 each independently ranges from about 0.1 cm to about 0.25 cm. In some embodiments, the ring body includes an inner wall 1241 and an outer wall 1242, wherein a thickness of the inner wall 1241 and the outer wall 1242 are each independently selected from a thickness of about 0.1 cm, 0.11 cm, 0.12 cm, 0.13 cm, 0.14 cm, 0.15 cm, 0.16 cm, 0.17 cm, 0.18 cm, 0.19 cm, 0.2 cm, 0.21 cm, 0.22 cm, 0.23 cm, 0.24 cm, or 0.25 cm. In some embodiments, the ring body includes an inner wall 1241 and an outer wall 1242, wherein a thickness of the inner wall 1241 and the outer wall 1242 each independently ranges from about 0.1 cm to 0.11 cm, 0.12 cm, 0.13 cm, 0.14 cm, 0.15 cm, 0.16 cm, 0.17 cm, 0.18 cm, 0.19 cm, 0.2 cm, 0.21 cm, 0.22 cm, 0.23 cm, 0.24 cm, or 0.25 cm.

In some embodiments, the ring body includes notch walls 1243, wherein a thickness of the notch walls 1243 ranges from about 0.1 cm to about 0.25 cm. In some embodiments, the thickness of the notch walls 1243 is about 0.1 cm, 0.11 cm, 0.12 cm, 0.13 cm, 0.14 cm, 0.15 cm, 0.16 cm, 0.17 cm, 0.18 cm, 0.19 cm, 0.2 cm, 0.21 cm, 0.22 cm, 0.23 cm, 0.24 cm, or 0.25 cm. In some embodiments, the thickness of the notch walls 1243 ranges from about 0.1 cm to 0.11 cm, 0.12 cm, 0.13 cm, 0.14 cm, 0.15 cm, 0.16 cm, 0.17 cm, 0.18 cm, 0.19 cm, 0.2 cm, 0.21 cm, 0.22 cm, 0.23 cm, 0.24 cm, or 0.25 cm.

The cricothyrotomy device 1000 or components thereof can be composed on one or more suitable materials. Suitable materials include, without limitation, metals, alloys, polymers, silicone, or any combination thereof. Thus, in some embodiments, the port 1100 and the ring lock 1200 each independently include or are solely composed of a metal, an alloy, a polymer, silicone, or any combination thereof. In some embodiments, the suitable material(s) are medical grade. In some embodiments, the suitable material(s) are medical instrument grade. In some embodiments, the suitable material(s) are surgical grade. Exemplary metals include, but are not limited to, aluminum, titanium, chromium, copper, gold, and silver. Exemplary alloys include, but are not limited to, stainless steel (including, but not limited to, 301/302, 303, 304, 304L, 316 VM, 316L, 420 Mod, 420A, 420B, 420C, 420F, 431, 431 (X), 440A, 440B, 440C, 630, Custom 465, and XM16 Custom 455 alloys) and cobalt alloys (e.g., MP35N alloy and Phynox alloy). Exemplary polymers include, without limitation, polycarbonate, polypropylene, polyethylene, polyvinylchloride, acrylonitrile butadiene styrene, polystyrene, polyethylene terephthalate glycol, polymethyl methacrylate, rubber (natural or synthetic), polyether ketone (PEEK), polyaryletherketone (PAEK), polyphenylsulfone (PPSU), polysulfone (PSU), polyacrylamide (PARA), and any combination thereof.

In some embodiments, the one or more suitable materials are biocompatible. In some embodiments, the one or more suitable materials are suitable for sterilization. In some embodiments, the one or more suitable materials are such that the cricothyrotomy device 1000 is configured for single use. In some embodiments, the one or more suitable materials are such that the cricothyrotomy device 1000 is configured for multiple uses. In some embodiments, the one or more suitable materials are radio translucent. In some embodiments, the one or more suitable materials are radio opaque. In some embodiments, the port 1100 and the ring lock 1200 each independently include or are composed solely of a radio translucent material or a radio opaque material.

The cricothyrotomy device 1000 or component thereof of the present disclosure can be made by any suitable manufacturing techniques which will be apparent to those of ordinary skill in the art. Such manufacturing techniques are generally known in the art, and include without limitation, molding (e.g., injection, compression, transfer, extrusion, blow, dip, rotational, thermoforming, laminating, vacuum, pressure, matched, shrink wrapping, etc.), three-dimensional manufacturing techniques (e.g., additive manufacturing, engraving, chemical vapor deposition, and lithography), casting (e.g., centrifugal, continuous, die, evaporative-pattern, investment casting, low-pressure die, permanent mold, sand, resin, shell molding, and slush or slurry casting), spraying, forming (e.g., end tube forming, forging, rolling, extrusion, pressing, bending, and shearing), machining (e.g., milling, grinding, turning, drilling, reaming, tapping, sawing, broaching, shaping, planning, honing, routing, hobbing, electrical discharge machining, gashing, and finishing), and joining (e.g., welding, brazing, soldering, sintering, bonding, fastening, and press fitting).

The cricothyrotomy device of the present disclosure can be used to perform a cricothyrotomy procedure. Thus, described in several exemplary embodiments herein is a method of performing a cricothyrotomy on a subject, the method including coupling the port of the cricothyrotomy device of the present disclosure to the ring lock of the cricothyrotomy device by inserting the tapered end of the port into the central opening of the ring lock and moving the ring lock to the base cannula portion of the port, and inserting the tapered end of the elongated cannula into a neck of the subject such that an airway is established. In some embodiments, coupling the port to the ring lock comprises screwing the ring lock screw thread of the ring lock onto the port screw thread. In some embodiments, insertion of the tapered end is through an incision in a cricothyroid membrane of the subject. In some embodiments, insertion of the tapered end forms an incision in the skin, muscle and/or other soft tissue (including, but not limited to, the cricothyroid membrane) of the subject. In some embodiments, the method further comprises inserting one or more guide wires through the opening of the cricothyrotomy device and into a trachea of the subject. In some embodiments, after the airway is established and optionally one or more guide wires placed, the cricothyrotomy device can be removed from the subject.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein.

### Example 1

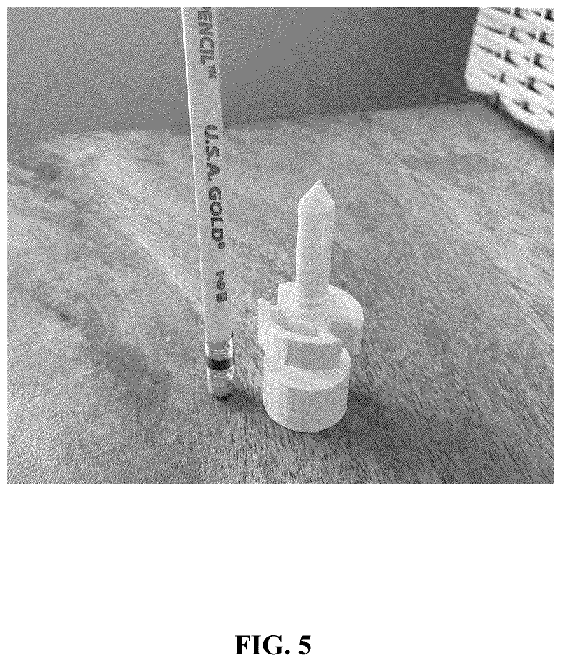

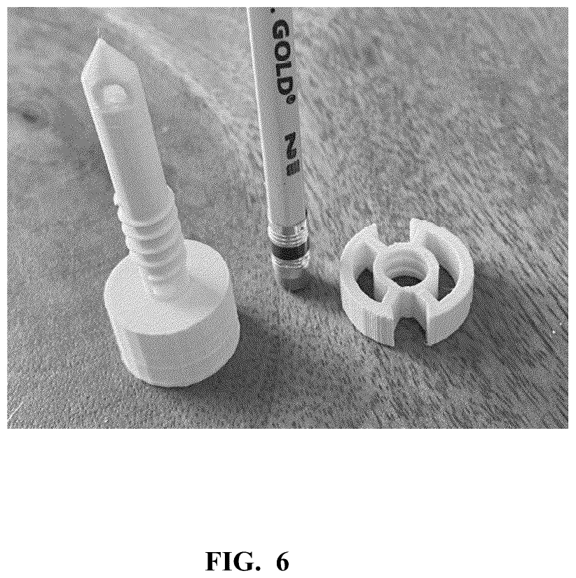

Applicant generated prototypes of the cricothyrotomy device according to several embodiments of the present disclosure. Images of an exemplary prototype cricothyrotomy device according to several embodiments of the present disclosure are shown in FIGS. 5-6. The prototype device was made from one or more polymers using standard molding and/or three-dimensional printing techniques.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features before set forth herein.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A cricothyrotomy device comprising:
a port comprising:
   an elongated cannula portion comprising a tapered end;
   a base cannula portion coupled to or integrated with the elongated cannula portion, wherein the base cannula portion is coupled to or integrated with the elongated cannula portion at the end of the elongated cannula portion opposite the tapered end;
   a port opening in the elongated cannula portion, tapered end, or extending into both, wherein the elongated cannula portion and the base cannula portion are coupled or integrated such that an opening extends from the port opening through the elongated cannula portion and base cannula portion;
   a port screw thread on the outer surface of the elongated cannula portion at the end of the elongated cannula portion opposite the tapered end and adjacent to the base cannula portion;
   one or more lock tabs, wherein the one or more lock tabs are positioned on an outer surface of the elongated cannula portion at the end of the port screw thread adjacent to the base cannula portion; and
a ring lock configured to couple with the port, the ring lock comprising:
   a ring body having a central opening;
   a ring lock screw thread on the surface of a wall of the ring body facing the central opening, wherein the ring lock screw thread is configured to couple with the port screw thread during use; and
   one or more notches configured to couple with the one or more lock tabs of the port during use.
      2. The cricothyrotomy device of aspect 1, wherein the port has a length (lₚ) and wherein the length of the port ranges from about 1 cm to about 6 cm.
      3. The cricothyrotomy device of any one of aspects 1-2, wherein the elongated cannula portion has a length (l_{ec}), wherein the length of the elongated cannula portion (l_{ec}) extends from a tip of the tapered end of the elongated cannula portion to the base cannula portion and wherein the length of the elongated cannula portion (l_{ec}) ranges from about 0.5 cm to about 5.5 cm (l_{ec}).
      4. The cricothyrotomy device of any one of aspects 1-3, wherein the opening has a diameter of about 0.1 cm to about 1.5 cm.
      5. The cricothyrotomy device of any one of aspects 1-4, wherein the port opening has a diameter of about 0.1 cm to about 1.5 cm.
      6. The cricothyrotomy device of any one of aspects 1-5, wherein a thickness of a wall of the elongated cannula portion ranges from about 0.1 cm to about 0.5 cm.
      7. The cricothyrotomy device of any one of aspects 1-6, wherein a length of the tapered end (lₜₑ) ranges from 0.1 cm to about 0.5 cm.
      8. The cricothyrotomy device of any one of aspects 1-7, wherein the port screw thread has a length (lₚₛₜ) ranging from about 0.5 cm to about 3 cm.
      9. The cricothyrotomy device of any one of aspects 1-8, wherein an outer diameter of the elongated cannula portion ranges from about 0.3 cm to about 2.5 cm.
      10. The cricothyrotomy device of any one of aspects 1-9, wherein the base cannula portion has a width (w_{bc}) ranging from about 0.25 cm to about 2 cm.
      11. The cricothyrotomy device of any one of aspects 1-10, wherein the base cannula portion has a length (l_{bc}) ranging from about 0.5 cm to about 3 cm.
      12. The cricothyrotomy device of any one of aspects 1-11, wherein a wall of the base cannula portion has a thickness ranging from about 0.3 cm to about 2.25 cm.
      13. The cricothyrotomy device of any one of aspects 1-12, wherein an outer diameter of the base cannula portion ranges from about 0.6 cm to about 6 cm.
      14. The cricothyrotomy device of any one of aspects 1-13, wherein an outer diameter of the ring lock (d_{odr1}) ranges from about 0.6 cm to about 6 cm.
      15. The cricothyrotomy device of any one of aspects 1-14, wherein a diameter of the central opening (d_{co}) of the ring lock ranges from about 0.3 cm to about 2.5 cm.
      16. The cricothyrotomy device of any one of aspects 1-15, wherein a width of the ring body ranges (w_{rb}) from about 0.1 cm to about 1.25 cm.
      17. The cricothyrotomy device of any one of aspects 1-16, wherein the ring body comprises an inner wall and an outer wall, wherein a thickness of the inner wall and the outer wall each independently ranges from about 0.1 cm to about 0.25 cm.
      18. The cricothyrotomy device of any one of aspects 1-17, wherein the ring body comprises notch walls, wherein a thickness of the notch walls ranges from about 0.1 cm to about 0.25 cm.
      19. The cricothyrotomy device of any one of aspects 1-18, wherein the port and the ring lock each independently comprise or consist of a metal, an alloy, a polymer, silicone, or any combination thereof.
      20. The cricothyrotomy device of any one of aspects 1-19, wherein the port and the ring lock each independently comprise or consist of a radio translucent material or a radio opaque material.
      21. A method of performing a cricothyrotomy on a subject, the method comprising coupling the port of the cricothyrotomy device of any one of aspects 1-20 to the ring lock of the cricothyrotomy device by inserting the tapered end of the port into the central opening of the ring lock and moving the ring lock to the base cannula portion of the port; and inserting the tapered end of the elongated cannula portion into a neck of the subject such that an airway is established.
      22. The method of aspect 21, wherein coupling the port to the ring lock comprises screwing the ring lock screw thread of the ring lock onto the port screw thread.
      23. The method of any one of aspects 21-22, wherein insertion of the tapered end is through an incision in a cricothyroid membrane of the subject or wherein insertion of the tapered end forms an incision in the cricothyroid membrane of the subject.
      24. The method of any one of aspects 21-23, further comprising inserting one or more guide wires through the opening of the cricothyrotomy device and into a trachea of the subject.

What is claimed is:

1. A cricothyrotomy device comprising:
a port comprising:
an elongated cannula portion comprising a tapered end;
a base cannula portion coupled to or integrated with the elongated cannula portion, wherein the base cannula portion is coupled to or integrated with the elongated cannula portion at the end of the elongated cannula portion opposite the tapered end;
a port opening in the elongated cannula portion, tapered end, or extending into both, wherein the elongated cannula portion and the base cannula portion are coupled or integrated such that an opening extends from the port opening through the elongated cannula portion and base cannula portion;
a port screw thread on the outer surface of an elongated cannula portion at the end of the elongated cannula portion opposite the tapered end and adjacent to the base cannula portion;
one or more lock tabs, wherein the one or more lock tabs are positioned on an outer surface of the elongated cannula portion at the end of the port screw thread adjacent to the base cannula portion; and
a ring lock configured to couple with the port, the ring lock comprising:
a ring body having a central opening;
a ring lock screw thread on a surface of a wall of the ring body facing the central opening, wherein the ring lock screw thread is configured to couple with the port screw thread during use; and
one or more notches configured to couple with the one or more lock tabs of the port during use.

2. The cricothyrotomy device of claim 1, wherein (a) the port has a length (lₚ) and wherein the length of the port ranges from about 1 cm to about 6 cm; (b) the port screw thread has a length (lₚₛₜ) ranging from about 0.5 cm to about 3 cm; or (c) both (a) and (b).

3. The cricothyrotomy device of claim 1, wherein the elongated cannula portion has a length (l_{ec}), wherein the length of the elongated cannula portion (l_{ec}) extends from a tip of the tapered end of the elongated cannula portion to the base cannula portion and wherein the length of the elongated cannula portion (l_{ec}) ranges from about 0.5 cm to about 5.5 cm (l_{ec}).

4. The cricothyrotomy device of claim 1, wherein the opening has a diameter of about 0.1 cm to about 1.5 cm.

5. The cricothyrotomy device of claim 1, wherein the port opening has a diameter of about 0.1 cm to about 1.5 cm.

6. The cricothyrotomy device of claim 1, wherein a thickness of a wall of the elongated cannula portion ranges from about 0.1 cm to about 0.5 cm.

7. The cricothyrotomy device of claim 1, wherein a length of the tapered end (lₜₑ) ranges from 0.1 cm to about 0.5 cm.

8. The cricothyrotomy device of claim 1, wherein an outer diameter of the elongated cannula portion ranges from about 0.3 cm to about 2.5 cm.

9. The cricothyrotomy device of claim 1, wherein (a) the base cannula portion has a width (w_{bc}) ranging from about 0.25 cm to about 2 cm; (b) the base cannula portion has a length (l_{bc}) ranging from about 0.5 cm to about 3 cm; or (c) a wall of the base cannula portion has a thickness ranging from about 0.3 cm to about 2.25 cm; or (d) any combination thereof.

10. The cricothyrotomy device of claim 1, wherein an outer diameter of the base cannula portion ranges from about 0.6 cm to about 6 cm.

11. The cricothyrotomy device of claim 1, wherein an outer diameter of the ring lock (d_{odr1}) ranges from about 0.6 cm to about 6 cm.

12. The cricothyrotomy device of claim 1, wherein a diameter of the central opening (d_{co}) of the ring lock ranges from about 0.3 cm to about 2.5 cm.

13. The cricothyrotomy device of claim 1, wherein a width of the ring body ranges (w_{rb}) from about 0.1 cm to about 1.25 cm.

14. The cricothyrotomy device of claim 1, wherein the ring body comprises an inner wall and an outer wall, wherein a thickness of the inner wall and the outer wall each independently ranges from about 0.1 cm to about 0.25 cm.

15. The cricothyrotomy device of claim 1, wherein the ring body comprises notch walls, wherein a thickness of the notch walls ranges from about 0.1 cm to about 0.25 cm.

16. The cricothyrotomy device of claim 1, wherein (a) the port and the ring lock each independently comprise or consist of a metal, an alloy, a polymer, silicone, or any combination thereof; (b) wherein the port and the ring lock each independently comprise or consist of a radio translucent material or a radio opaque material; or (c) both (a) and (b).

17. A method of performing a cricothyrotomy on a subject, the method comprising
coupling the port of the cricothyrotomy device of claim 1 to the ring lock of the cricothyrotomy device by inserting the tapered end of the port into the central opening of the ring lock and moving the ring lock to the base cannula portion of the port; and
inserting the tapered end of the elongated cannula portion into a neck of the subject such that an airway is established.

18. The method of claim 17, wherein coupling the port to the ring lock comprises screwing the ring lock screw thread of the ring lock onto the port screw thread.

19. The method of claim 17, wherein insertion of the tapered end is through an incision in a cricothyroid membrane of the subject or wherein insertion of the tapered end forms an incision in the cricothyroid membrane of the subject.

20. The method of claim 17, further comprising inserting one or more guide wires through the opening of the cricothyrotomy device and into a trachea of the subject.
